(19) United States
(12) Patent Application Publication
(10) Pub. No.: US 2024/0058278 A1
(43) Pub. Date: Feb. 22, 2024

(54) Treatment for Primary or Secondary Refractory Chronic Insomnia and/or Other Diagnosed Issues Related to Sleep Deprivation

(71) Applicant: Nyree Penn, Parkland, FL (US)

(72) Inventor: Nyree Penn, Parkland, FL (US)

(21) Appl. No.: 18/236,680

(22) Filed: Aug. 22, 2023

Related U.S. Application Data
(60) Provisional application No. 63/399,754, filed on Aug. 22, 2022.

(51) Int. Cl. A61K 31/05

(52) U.S. Cl. CPC A61K 31/05

(57) ABSTRACT

A method of treatment for individuals suffering from chronic primary or secondary refractory chronic insomnia by administration of Diprivan/Propofol, to move a patient through the stages of sleep such that the patient obtains sufficient adequate rest.

### BACKGROUND

### Cross-Reference to Related Applications

This application is related to, and claims the benefit of, the provisional patent application entitled “Treatment for Primary or Secondary Refractory Chronic Insomnia and/or Other Diagnosed Issues Related to Sleep Deprivation”, filed Aug. 22, 2022, bearing U.S. Ser. No. 63/399,754 and naming Nyree Penn, the named inventor herein, as sole inventor, the contents of which is specifically incorporated by reference herein in its entirety.

### Technical Field

This invention relates in general to sleep disorders. In particular, it relates to a method of treatment for individuals suffering from sleep deprivation disorders.

### Background of the Invention

Many individuals have serious health problems related to sleep disorders. It is known that restorative sleep is essential to a person's health, and that sleep is a biological necessity. If an individual has chronic insufficient sleep due to untreated sleep disorders it can be detrimental for health, wellbeing, and public safety. Neurologically, sleep allows the brain to renew energy and remove waste and toxins from the brain. This process is important for memory consolidation and improving efficiency. From a mental health perspective, sleep disturbance is the most common prodrome of mania and the sixth most common prodrome of depression. Sleep loss is highly correlated with daily manic symptoms and negative affect. For human health, there is a direct relationship between sleep and health.

Several co-morbid medical conditions can contribute to insomnia. These include obstructive sleep apnea (OSA), fibromyalgia, restless legs syndrome (RLS), cardiovascular diseases, diabetes, arthritis, migraine, asthma, chronic obstructive pulmonary disease, and chronic pain. Therefore the necessity to get restorative sleep is not only imperative, but, directly correlated to an individual's mortality. It would be desirable to have a method of improving the type, amount, and quality of sleep available to an individual.

While the prior art has provided a variety of pharmaceutical treatment methods for acute and chronic insomnia, it has failed to provide a treatment that is effective for individuals suffering from severe sleep deprivation disorders.

### SUMMARY OF THE INVENTION

This invention provides a method of treatment for primary and/or secondary refectory chronic insomnia and/or other diagnosed issues related to sleep deprivation. The treatment method includes a medically supervised administration of Diprivan/Propofol to quickly and temporarily move a patient through the sleep cycles to alleviate chronic sleep deprivation health problems.

### BRIEF DESCRIPTION OF THE DRAWINGS

FIG. 1 is a chart that illustrates the results of an EEG measurement.

### DETAILED DESCRIPTION OF THE PREFERRED EMBODIMENT

Prior to a detailed description on the FIGURES, a general overview of the invention will be presented.

The drug Diprivan/Propofol is used as an anesthetic drug for surgery. This invention discloses a new use for the anesthetic drug. Diprivan/Propofol which provides a single effective treatment for primary or secondary refractory chronic insomnia. The invention is directed to helping individuals suffering from sever sleep disorders. Many individuals have serious health problems related to sleep disorder, especially when those individuals are not able to have the benefits of restorative sleep. It is known that restorative sleep is essential to a person's health, and that sleep is a biological necessity. If an individual has chronic insufficient sleep due to untreated sleep disorders it can be detrimental for health, wellbeing, and even safety for themselves and others. Neurologically, sleep allows the brain to renew energy and remove waste and toxins from the brain. This process is important for memory consolidation and improving efficiency. From a mental health perspective, sleep disturbance is the most common prodrome of mania and the sixth most common prodrome of depression. Sleep loss is highly correlated with daily manic symptoms and negative affect. For human health, there is a dependent, bidirectional, co-dependent relationship between sleep and health.

As noted above, several co-morbid medical conditions, such as sleep apnea (OSA), fibromyalgia, restless legs syndrome (RLS), cardiovascular diseases, diabetes, arthritis, migraine, asthma, chronic obstructive pulmonary disease, and chronic pain can prevent an individual from getting the necessary amount of restorative sleep that is directly correlated to an individual's health and mortality.

Further, while individuals need an adequate amount of sleep in general, individuals also need specific types of sleep for good health, because not all sleep framework and physiology operate in the same manner. It is this physiological difference that determines the “restfulness” of a person. Individuals experience sleep in a cyclic manner that consists of stages, and within each stage, there am two primary forms of sleep: rapid eye movement (REM) sleep and non-rapid eye movement NREM) sleep, in which the “depth of sleep” is determined. The depth of sleep can be measured by brain wave activity on an electroencephalogram (EEG). An EEG is a measurement of brain activity in which each stage of sleep presents different EEG waves. The deeper in the stage of sleeping, the more amplitude with less frequency of EEG wave changes occur.

In addition to the amount of sleep, individuals require adequate time in each of the several types of sleep. Sleep typically begins with the NREM phase, which is followed by the REM phase. NREM sleep is subdivided into three stages: N1, N2, and N3, in which brain activity, eye movement and skeletal muscle tone progressively decreases, each stage placing the individual in a deeper state of sleep. Later in the cycle, when a person becomes “lighter in sleep”, the individual enters REM sleep.

In addition, in the human sleep cycle, discussed above, it is important to understand that sleep cycles vary. Most people experience four to six “sleep cycles” throughout the night.

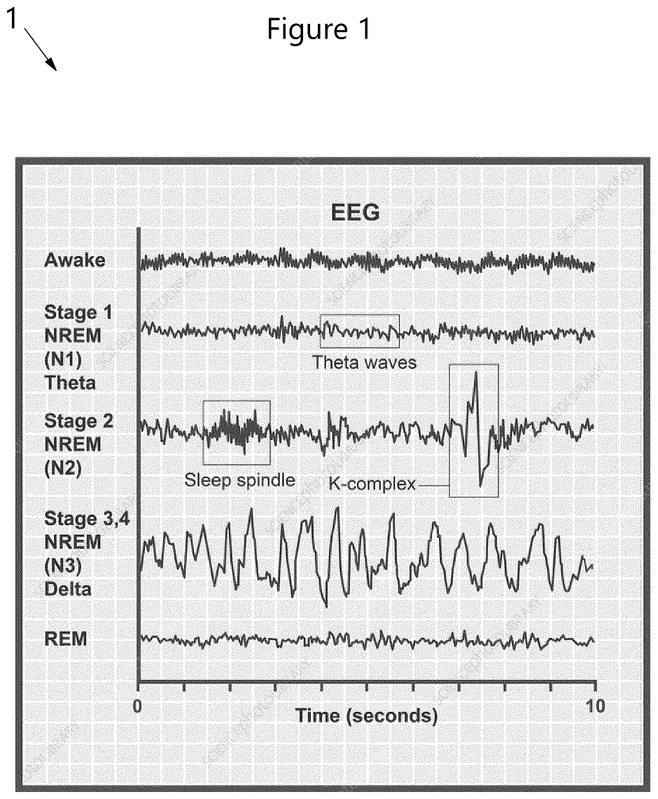

Stages of NREM sleep as illustrated in FIG. 1:
1. N0/NREM Sleep
   Stage 0/REM sleep: Awake and relaxed to drowsy. EEG shows the presence of Alpha and Beta waves with low amplitude and mixed frequency waves. The duration is 1-5 minutes initially, 15% of total sleep time. Physiologically, electrical activity in the brain starts to slow, contributing to decreased blood flow to the brain, decrease in heart rate (HR) and respiratory rate (RR) correlate with physical changes that correlate with activities of the hypothalamic-pituitary-adrenal (HPA) axis and the sympathetic nervous system in healthy humans. The waves lengthen with each successive cycle as a person enters into the next stage of sleep.
2. N1/NREM Sleep
   In the beginning stage of N1/NREM sleep, it has a short duration, typically 1-7 minutes, 2-5% of total sleep time. Physiologically, there is a decrease in heart rate (HR), respiratory rate (RR) and rapid eye movement (REM) slows as muscles relax with relatively few twitches. This is considered, light sleep that is easily interrupted by noises or disturbances.
3. N2/NREM Sleep
   In second stage N2/NREM sleep, an EEG shows the presence of advanced Theta waves with sleep spindles and K-complexes appearing before the next transitional stage of sleep. The presence of these type of wave morphologies are important indicators that response to stimulus is suppressed. The duration is typically 10-25 minutes, 45-55% of total sleep time. Physiologically there is a further decrease in HR. RR and rapid eye movement slows as muscles relax with relatively no twitches and body temperature decreases. Considered moderate sleep with greater depth.
4. N3/NREM Sleep
   N3/NREM is the third stage of NREM sleep, and is the deepest state of sleep. In N3/NREM sleep, the EEG shows the presence of Delta waves with high amplitude and low frequency waves. The duration is 20-40 minutes, 15% of total sleep time. Physiologically, HR, RR are at their lowest levels during sleep in this stage. Eye-movements stop, muscles relax even further and body temperature continues to decrease. For the purposes of this invention, it is during this stage that anesthesia is targeted and maintained. However, the stages of N2 and N3 often merge during sleep cycles. It is at this stage where restorative, deep sleep that is needed to feel restored and refreshed happens. The awakening threshold for disturbances is highest during this stage. The cyclic process of sleep will continue with the return to the REM stage.

The process and regulation of entering into and out of the various stages of REM and NREM sleep is facilitated by the neurotransmitters gamma-aminobutyric acid (GABA) and histamine. In the forebrain and hypothalamus, neurons release GABA and histamine. These neurotransmitters have opposing actions on the sleep-wake cycle. Increased GABA and decreased histamine release induce NREM sleep by deactivating the cortex and thalamus. The sleep-wake cycle is also affected by neurotransmitters released by reticular activating system (RAS) neurons, such as norepinephrine, acetylcholine, and serotonin. These neurotransmitters contribute to maintaining wakefulness and significantly decrease during REM sleep. Orexin, which is produced in the hypothalamus, is a neuropeptide that plays an important ole in maintaining wakefulness. It is hypothesized that the action of orexin changes the activity of the neurotransmitters involved in the regulation of sleep/wake states. Melatonin is a hormone that plays an integral role in diurnal rhythms. It synchronizes the body with the environment's light-dark cycle, peaking during the night and dipping during the day, to stabilize the body's natural circadian rhythm.

The prior art recommended sequence of medication protocols for treatment include: Short or-intermediate-acting benzodiazepine receptor agonists (BzRAs) or the melatonin agonist ramelteon. Alternative short or intermediate-acting BzRAs or ramelteon if the initial agent was ineffective. Sedating antidepressants (e.g., trazodone, amitriptyline, doxepin, or mirtazapine). The combination of a BzRA or ramelteon with a sedating antidepressant. Other sedating agents such as antiepilepsy medications tor atypical antipsychotics. Of note, the prior art protocol requires a sequela of medication combinations. If the current protocol were effective, there would not be a need for the additional protocols and procedures that incorporate the mixture of other, potentially harmful medications. The outcome is and has been a subsequent addictive, unsuccessful result with little or no options left for the patient. The reason for this extensive protocol of “trial-and-error” methods to treat insomnia is due to the inability of those drugs to adequately reach the GABA receptors in the brain and maintain the depth of N3/NREM sleep state necessary for restorative sleep. This is why the current pharmaceutical treatment methods for acute and chronic insomnia are numerous, yet ineffective because the prior art methods do not appropriately target the neurotransmitter receptor, GABAA.

The action of Diprivan/Propofol involves a positive modulation of the inhibitory function of the neurotransmitter gamma-aminobutyric acid (GABA) through GABAA receptors. This action increases GABA-mediated inhibitory tone in the CNS by decreasing the rate of dissociation of the GABA from the receptor. This increase in the duration of the GABA-activated opening of the chloride channel will result in hyperpolarization of cell membranes. And at clinical concentrations, it directly activates the receptor's chloride channel, thereby inducing controlled sleep of an anesthetized person.

The invention overcomes this problem by using a Diprivan/Propofol derivative. Diprivan/Propofol is an FDA approved, short acting, intravenous, sterile emulsion. It is currently available in the United States as a prescription, anesthetic drug for the use in human and veterinary medicine. Diprivan/Propofol is a non-barbiturate sedative, used in hospital settings by trained anesthetists for the induction, maintenance of general anesthesia and sedation of ventilated adults receiving intensive care, for a period of up to 72 hours.

The instant invention is a new use of Diprivan/Propofol as a non-surgical treatment for primary or secondary refractory chronic insomnia and/or other diagnosed issues related to sleep deprivation. It binds post-synaptically and enhances GABAergic inhibition, counteracting arousal inputs to the pyramidal neuron, decreasing its excitatory activity, and provides the contributing physiological result of sleep and unconsciousness.

As used in the instant invention, the unique chemical and physiological properties of Diprivan/Propofol deactivates the GABAA receptors to allow the patient to reach N2/N3 of restorative sleep states. Additionally, a further advantage of Diprivan/Propofol is that it provides an ultra-fast onset and offset times, usually causing sleep within seconds of injection. Because the drug is short-acting, it is often administered by a continuous intravenous drip or infusion. And, because Diprivan/Propofol is short-acting, the patient usually awakens within minutes of discontinuing the drug. It has been found that using Diprivan/Propofol is advantageous in that the sleep cycle begins very quickly at onset of the treatment, and wry quickly stops acting at the end of the treatment. This is an important advantage of the invention in that it allows individuals suffering from sleep disorders to quickly begin the process of passing from sleep cycles, and then quickly recover in to wide awake and rested status at the end of the treatment, so that a maximum amount of rest and recovery from sleep deprivation can be achieved with a minimum amount of patient time. Due to the reliance of the medical industry on prior art pharmaceutical products, this treatment protocol has heretofore not been available to patients.

In the preferred environment this method of treatment will be administered in comfortable, designated sleep quarters, located within an outpatient, clinical sleep facility. During each one-hour sleep session, the patient will have all vital recordings monitored in accordance with the American Society of Anesthesiologists Physical Status (ASA). A licensed Anesthesiologist (MDA) and/or Certified Anesthesiologist Assistant (CAA) or Certified Nurse Anesthetist (CRNA), who is extensively trained in airway management and airway techniques, will administer the Diprivan/Propofol intravenously to the patient.

The dosage administered to the patient will be determined by the medical professional based on the patient's individual health factors, such as age, weight, preexisting medical conditions, etc.

This method of treatment is the only intravenous pharmaceutical agent for use in the treatment of primary or secondary refractory chronic insomnia and/or other diagnosed issues related to sleep deprivation. The preferred embodiment envisions that each patient will have a recorded one-hour sleep session in which the trained anesthesia professional will be physically present in the sleeping quarters with the patient at all times. In addition, the one-hour sleep session will follow the same local, state, federal and medication guidelines for the safe delivery of monitored anesthesia care (MAC).

During the sleep session, the trained anesthesia provider will pay attention to the patient and monitor any cardiorespiratory effects, hypotension, oxyhemoglobin desaturation, signs of apnea and/or airway obstruction. During initiation of MAC anesthesia with Diprivan/Propofol, slow infusion, slow injection techniques or intermittent bolus administration will be based on the clinical judgement of the anesthesia clinician. The American Society of Anesthesiologists Physical Status (ASA-PS) I thru IV status will be determined upon the patient's pre-procedure consultation.

In the preferred embodiment, each one-hour sleep session is broken down in minutes, as follows:
a. 1-15 minutes: pre-procedure consultation where ASA status will be determined (outside of sleep quarters).
b. 16-20 minutes: allowing the patient to “get comfortable” (inside of sleep quarters).
c. 21-40 minutes: 20-minutes of Diprivan/Propofol via slow infusion, slow injection techniques or intermittent bolus administration (to be determined by anesthesia provider). A constant Diprivan/Propofol infusion allows an N3 sleep cycle to be maintained in a fixed state of NREM restorative sleep.
d. 41-45 minutes: to allow the patient to “wake-up”.
e. 46-0 minutes: post-procedure recovery—upon completion of the sleep-session, the cam of the patient will be transferred to the outpatient recovery area (outside of sleep quarters).

Having discussed the features and advantages of the invention in general, we turn now to a discussion of the FIGURES.

FIG. 1 shows the results an EEG measurement 1 which illustrates the various wave patterns that occur in particular states of sleep.

Stages of NREM sleep:
1. N0/NREM Sleep
   Stage 0/REM sleep is syndicated by the line titled “Awake.” This is the state when an individual is relaxed to drowsy. The EEG shows the presence of Alpha and Beta waves with low amplitude and mixed frequency waves. Physiologically, electrical activity in the brain starts to slow, contributing to decreased blood low to the brain, decrease in heart rate (HR) and respiratory rate (RR) correlate with physical changes that correlate with activities of the hypothalamic-pituitary-adrenal (HPA) axis and the sympathetic nervous system in healthy humans. The waves lengthen with each successive cycle as a person enters into the next stage of sleep.
2. N1/NREM Sleep
   The second line is titled “Stage 1 NREM (N1) Theta.” It illustrates the N1/NREM stage of sleep as measured by an EEG. Physiologically, there is a decrease in heart rate (HR), respiratory rate (RR) and rapid eye movement (REM) slows as muscles relax with relatively few twitches. This is considered, light sleep that is easily interrupted by noises or disturbances.
3. N2/NREM Sleep
   The third line is titled “Stage 2 NREM (N2).” In this stage of N2/NREM sleep, an EEG shows the presence of advanced Theta waves with sleep spindles and K-complexes appearing before the next transitional stage of sleep. The presence of these type of wave morphologies are important indicators that response to stimulus is suppressed. Physiologically there is a further decrease in HR, RR and rapid eye movement slows as muscles relax with relatively no twitches and body temperature decreases. Considered moderate sleep with greater depth.
4. N3/NREM Sleep
   The Fourth line is titled “Stage 3, 4 NREM (N3) Delta.” N3/NREM is the third stage of NREM sleep and is the deepest state of sleep. In N3/NREM sleep, the EEG shows the presence of Delta waves with high amplitude and low frequency waves. Physiologically, HR, RR are at their lowest levels during sleep in this stage. Eye-movements stop, muscles relax even further and body temperature continues to decrease. It is during this stage that anesthesia is targeted and maintained. However, the stages of N2 and N3 often merge during sleep cycles or under anesthesia. It is at this stage where restorative, deep sleep that is needed to feel restored and refreshed happens. The awakening threshold for disturbances is highest during this stage. The cyclic process of sleep will continue with the return to the REM (light sleep) stage.
5. REM Sleep
   The fifth line is titled “REM” and illustrates the lights sleep stage as measured by an EEG.

While the preferred method of treatment disclosed herein have been discussed to illustrate the invention, it will be understood by those skilled in the art that variations can be made without departing from the spirit of the invention. The frequency of treatment can vary, the method of administering the treatment, such as slow infusion, slow injection techniques or intermittent bolus administration will be based on the clinical judgement of the anesthesia clinician can vary, etc. Therefore, the invention shall be limited solely to the scope of the claims.

I claim:

1. A method of treating primary or secondary refractory chronic insomnia related to sleep deprivation, comprising:
administering Diprivan/Propofol to a patient under control of a trained medical professional;
using a sufficient amount of Diprivan/Propofol to progress the patient through the NREM sleep stages such that the patient is placed in N3/NREM sleep;
after a predetermined amount of sleep time, stopping the administration of Diprivan/Propofol such that the patient awakes.

2. A method, as in claim 1, wherein a trained medical professional consults the patient prior to administration of Diprivan/Propofol to determine the appropriate dosage of Diprivan/Propofol based on the patient's individual health profile.

3. A method, as in claim 2, wherein consultation with the patient takes approximately 1 to 15 minutes.

4. A method, as in claim 2, wherein sleep quarters are provided for the patient prior to administration of Diprivan/Propofol.

5. A method, as in claim 4, wherein the patient is allowed sufficient time to become comfortable in the sleep quarters.

6. A method, as in claim 5, wherein time for the patient to become comfortable in the sleep quarters takes approximately 1 to 4 minutes.

7. A method, as in claim 6, wherein the administration of Diprivan/Propofol is done by a medical professional.

8. A method, as in claim 7, wherein the administration of Diprivan/Propofol takes approximately 40 minutes.

9. A method, as in claim 8, wherein the administration of Deprival/Propofol takes approximately 21 to 40 minutes.

10. A method, as in claim 9, wherein the Diprivan/Propofol is administered via slow infusion.

11. A method, as in claim 9, wherein the Diprivan/Propofol is administered via slow injection techniques.

11. A method, as in claim 9, wherein the Diprivan/Propofol is administered via intermittent bolus administration.

12. A method, as in claim 1, wherein the patient is awakened after the administration of Diprivan/Propofol is stopped when the predetermined amount of sleep time is reached.
